(19) United States
(12) Patent Application Publication
(10) Pub. No.: US 2005/0125847 A1
(43) Pub. Date: Jun. 9, 2005

(54) Use of coral red fluorescence proteins as tracers for easy identification of genetic modified Baculoviruses

(75) Inventors: Tzong-Yuan Wu, Panchiao City (TW); Feng-Ming Yang, Tainan City (TW); Suey-Sheng Kao, Taipei City (TW); Tzyy-Rong Jinn, Nan Tou City (TW)

Correspondence Address: Richard L. Byrne, 700 Koppers Building, 436 Seventh Avenue, Pittsburgh, PA, 15219-1818, US

(73) Assignee: CHUNG YUAN CHRISTIAN UNIVERSITY

(21) Appl. No.: 10/995,708

(22) Filed: Nov. 23, 2004

(30) Foreign Application Priority Data
Dec. 9, 2003 (TW) 92134744

(51) Int. Cl. A01K 67/00; A01K 67/033; A01N 63/00

(52) U.S. Cl. 800/8; 424/93.2; 424/93.6

(57) ABSTRACT

The present invention provides a method of tracking the presence of genetic modified baculoviruses (GMBVs) in pest insects, comprising infecting the pest insects with GMBVs, which are engineered to express tracer proteins, i.e., coral red fluorescence proteins. The color of the expressed coral red fluorescence proteins are red or pink and are bright enough to be seen by naked eyes under direct sunlight, thereby enabling the pest insects that are infected with GMBVs to be easily distinguished from the uninfected ones.

### RELATED APPLICATIONS

The present application is based on, and claims priority from, Taiwan Application Serial Number 92134744, filed Dec. 9, 2003, the disclosure of which is hereby incorporated by reference herein in its entirety.

### BACKGROUND

1. Field of Invention

The present invention relates to the use of coral red fluorescence protein as tracer for easy identification of genetic modified baculoviruses (GMBVs). More particularly, the present invention relates to a method of identifying GMBVs by use of coral red fluorescence proteins, which are co-expressed in GMBVs as tracers, thereby enabling the pests that are infected with GMBVs to be easily identified by naked eyes.

2. Description of Related Art

Traditionally pest control has been dominated by the use of chemical insecticides. Although they are fast acting, these chemicals are sometimes environmentally unattractive. In addition, many chemicals used in insect pest control are not species-specific and may affect non-target animals as well as the target pest. Furthermore, these chemicals or their by-products can sometimes persist in the environment for long periods of time.

Biological control, the use of living organisms to control insect pests, has become increasingly more acceptable as a means for controlling pests successfully. For example, the bio-insecticide Bacillus thuringiensis (Bt), is used for control of spruce budworm (see U.S. Pat. Nos. 5,061,489, and 5,039,523). However, some recent concerns over the specificity of Bt have resulted in the recommendation that it not be used in areas where there are endangered Lepidoptera. Ecological interests have resulted in a shift in emphasis to examine and develop other microbial products, including the insect viruses.

Insect viruses, such as Baculoviruses, are naturally occurring insect pathogens that are considered to be host specific and environmentally safe. They can persist for years to impact on several generations of insects. Baculoviruses are a large group of insect viruses that are known to infect over 500 different insect species, mainly Lepidoptera. Some baculoviruses infect insects which are pests of commercially important agricultural and forestry crops. Such baculoviruses are potentially valuable as biological control agents. There are sixteen countries using baculoviruses to control Lepidoptera and more than 30 species of baculoviruses have been developed as microbial insecticides (Moscardi, F., (1999) Annu. Rev. Entimol. 44, 257).

Baculovirus subgroups include nuclear polyhedrosis viruses, now called nucleopolyhedroviruses (NPVs) and granulosis viruses, now called granuloviruses (GVs). In the occluded forms of baculoviruses, the virions (enveloped nucleocapsids) are embedded in a crystalline protein matrix. This structure, referred to as an occlusion body, is the form found extraorganismally in nature, and it is generally responsible for spreading the infection between insects. The characteristic feature of the NPVs is that many virions are embedded in each occlusion body, which is relatively large (up to 5 micrometers). Occlusion bodies of single nucleopolyhedrosis viruses (SNPVs) are smaller and contain a single virion with multiple nucleocapsids each. Multiple nucleopolyedrosis viruses (MNPVS) have multiple nucleocapsids per virion and multiple virions per occlusion body. Granulosis viruses (GVs) have a single virion with one nucleocapsid per occlusion body. In nature, infection is initiated when an insect ingests food contaminated with baculovirus particles, typically in the form of occlusion bodies. The occlusion bodies dissociate under the alkaline conditions of the insect midgut, releasing the virions, which then invade epithelial cells lining the gut. Pre-occlusion bodies are also infective (see WO 97/08297, published Mar. 6, 1997). Within a host cell, the baculovirus migrates to the nucleus where replication takes place. Initially, specific viral proteins are produced within the infected cell via the transcription and translation of so-called “early genes.” Among other functions, these proteins are required for the replication of the viral DNA, which begins 4 to 6 hours after virus enters the cell. Viral DNA replication proceeds up to about 24 hours post-infection (pi). From about 8 to 24 hours pi, infected cells express “late genes” at high levels. These include components of the nucleocapsid that surround the viral DNA during the formation of progeny virus particles. Production of progeny virus particles begins around 12 hours pi. Initially, progeny viruses migrate to the cell membrane where they acquire an envelope as they bud out from the surface of the cell and are then called budding viruses. The nonoccluded, budding viruses can then infect other cells within the insect. Polyhedrin synthesis begins approximately 18 hours after infection and increases to very high levels by 24 to 48 hours pi. At about 24 hrs pi, there is a decrease in the rate of nonoccluded viruses production, and most progeny virus particles are then embedded in occlusion bodies. Occlusion body formation continues until the cell dies or lyses. Some baculoviruses infect virtually every tissue in the host insect so that at the end of the infection process, the entire insect is liquified, releasing extremely large numbers of occlusion bodies which can then spread the infection to other insects.

One problem associated with several natural insect virus as insecticide is that there is a time delay between the viral entry into the insect body and the lethal infection. Insect viruses must be ingested by larvae to allow infection. Occlusion bodies containing virus particles contaminating the foliage are eaten and dissolved by the insect's midgut juices, releasing virus particles. These particles pass through the gut cells and infect tracheal and other body tissues of the host larva. Over a period of 7 to 10 days, the virus replicates in susceptible 10 tissues eventually causing death. Infected larvae still feed, during this time; however, and hence significant defoliation of plants still can occur in the time interval between ingestion of virus and insect death. This feeding damage is an inherent problem with the use of natural insect viruses as pesticide.

The development of biotechnology provides tools to genetically modify insect viruses to enhance their efficacy and to relieve the feeding damage. Genes encoding toxins (scorpion and/or mite toxin), enzymes juvenile hormone (JH) esterase), neuropeptides (prothoracicotropic hormone), and eclosion hormone have been introduced into the viral genome by various research groups (Bonning and Hammock (1996) Annu. Rev. Entomol. 41:191-280). These genes encode secretary proteins or peptides which assert their functions outside of virus infected cells. Inserting the JH esterase gene into the Autographa Californica multiple capsid nucleopolyhedrovirus (AcMNPV) results in the secretion of the enzyme JH esterase into the hemolymph and improves the virus as a control agent. Several insect-specific toxins from scorpions and other insect predators have also been described and/or inserted into AcMNPV (See, e.g., EP 505,207; Maeda et al., (1991) Virology 184:777-780; Stewart et al., (1991) Nature 352:85-88). These proteins are neurotoxins that are secreted into the hemolymph and act on the nervous system. However, public concern is raised regarding the possible damage to the ecological system and/or human health if these genetic modified viruses containing toxin genes were released into the field (Maeda, S. (1995) Curr. Opin. Biotechnol. 6:313).

In view of the forgoing reasons, there exists a need for developing a method for easy identification and/or tracking these GMBV that act as insecticides.

### SUMMARY

As embodied and broadly described herein, the invention addresses the current tracking problem of GMBVs by co-expression tracer proteins in toxin gene included GMBV, the color of said tracer proteins are bright enough to be seen by naked eyes under direct sunlight, thereby enabling the infected pest insects being easily distinguished from those unaffected pest insects. Hence, the method according to this invention is useful in easing the public concerns of the consequences if the toxin gene included GMBVs were released into the field.

It is therefore an objective of the present invention to provide a method of tracking the presence of GMBVs in pest insects, comprising infecting the pest insects with GMBVs, which have been engineered to express tracer proteins, i.e., coral red fluorescence proteins. The expressed coral red fluorescence proteins are red or pink in color and are bright enough to be seen by naked eyes under direct sunlight thereby enabling the pest insects that are infected with GMBVs to be easily distinguished from the uninfected ones.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a flow chart describing the method of preparing the transfer vectors for construction of GMBVs that expressed DsREDs as tracer proteins according to this invention;

FIG. 2 illustrates the construction the bicisctronic DNA constructs (i.e., pBacDR-IR-GFP) containing dual fluorescence proteins of DsRED and EFGP of Example 1 of this invention;

FIG. 3 illustrates insect SF9 cells infected with vBacDR-IR-GFP under fluorescence microscope at Rhodamine channel (A) and FITC channel (B);

FIG. 4 illustrates T. ni larvae infected with vBacDR-IR-GFP and vAcp10-G (each larvae injected 4 ul virus solution with 1×10⁸ pfu/ml) under ultraviolet light (A) and visible light (B); and

FIG. 5 illustrates Spodoptera litura larvae (A), Plutella xylostella (B), and Spodoptera exigua larvae (C) infected with vBacDR-IR-GFP (4 ul virus solution, with 1×10⁸ pfu/ml) under visible light. All photos were taken on the 6th day after virus inoculation.

### DESCRIPTION OF THE PREFERRED EMBODIMENTS

In view of the foregoing tracking problem associated with toxin gene included GMBVs, this invention provides a method of tracking the presence of GMBVs in pest insects, comprising the step of infecting the pest insects with GMBVs, wherein said GMBVs have been engineered to express coral red fluorescence proteins as tracers. The expressed tracer proteins are either red or pink in color and are bright enough to be seen by naked eyes under direct sunlight without the aid of any prosthetic tool.

According to one embodiment of this invention, the GMBVs were engineered to produce two tracer proteins, i.e., the enhanced green fluorescence proteins (EGFPs) from Aequorea Victoria and coral red fluorescence proteins (DsREDs) from the non-bioluminescent coral Discosoma sp. for identification of the GMBVs infected pest insects. The expression of engineering gene(s) in a host cell is well known to any ordinary skilled person in the relevant art. EGFP is a standard reporter gene in molecular biology studies because no substrates or co-factors are needed and further because of its intrinsic bright, visible fluorescence derives from an internal fluorophore within the protein structure upon excitation with blue light (Kendall, J. M., and Badminton, M. N., (1998) Trends Biotechnol. 16:216-224.). Considerable efforts have been applied to create EGFP mutants with distinct spectral properties so as to generate multicolor image; and EFGP with blue, cyan, and yellow emissions are now available, but none of these fluorescence proteins emits above the wavelength of 529 nm (Baird et al., (2000) Proc. Natl. Acad. Sci. USA 97:11984-11989). As to coral red fluorescent proteins, they are cloned from the non-bioluminescent coral Discosoma sp. (Matz et al., (1999) Nat. Biotechnol. 17:969-973) with an excitation peak at 558 nm and an emission peak at 583 nm. In one embodiment of this invention, both EGFP and DsRED proteins are co-expressed in GMBV by use of bicistronic DNA transfection vectors containing IRES sequences of emcephalomyocardities viruses (EMCV-IRES). The IRES of EMCV has been wildly used in bicistronic expression vectors of mammalian cells (Dirks et al., (1993) Gene 128:247-249), thereby both EGFP and DsRED proteins are transcribed into same mRNA molecule and may subsequently be translated simultaneously.

According to one embodiment of this invention, insect larvae were infected with GMBVs that expressed both DsRED and EGFP proteins as described above, the red fluorescence emitted by DsRED was bright enough to be seen by naked eyes in visible light, whereas the green fluorescence emitted by EGFP was barely seen under direct sunlight. This observation has been further confirmed in another embodiment of this invention. In this particular embodiment, insect larvae were infected with GMBVs that were engineered to produce only one tracer protein, i.e., EGFPs. Similarly, the green fluorescence emitted by EGFPs can only be seen under ultraviolet light, but are not under direct sunlight. In fact, the fluorescence were so faint that infected larvae cannot be distinguished easily from the uninfected ones by naked eyes in visible light. This phenomenon renders DsRED a much better tracer protein than EGFP because of its visibility by naked eyes in visible light, and therefore, a more powerful tracer protein for tracking the presence of GMBVs in the infected larvae.

The infection of pest insects with GMBVs can be achieved via various routes, which includes, but are not limited to microinjecting, feeding, and/or spraying of a virus fluid containing GMBVs to the pest insects. According to one embodiment of this invention, infection was achieved by microinjection, though other routes may also be used. Among these routes, spray infection or aerosol infection, is most preferred. The spray infection method was disclosed in a co-pending Taiwan patent application No.: 92,127,510 filed by the applicants of this invention on Oct. 3, 2003. Briefly, the method comprises the steps of: providing a plurality of insect larvae; providing a virus fluid that contains GMBVs; and spraying the plurality of insect larvae with the virus fluid for aerosol infection.

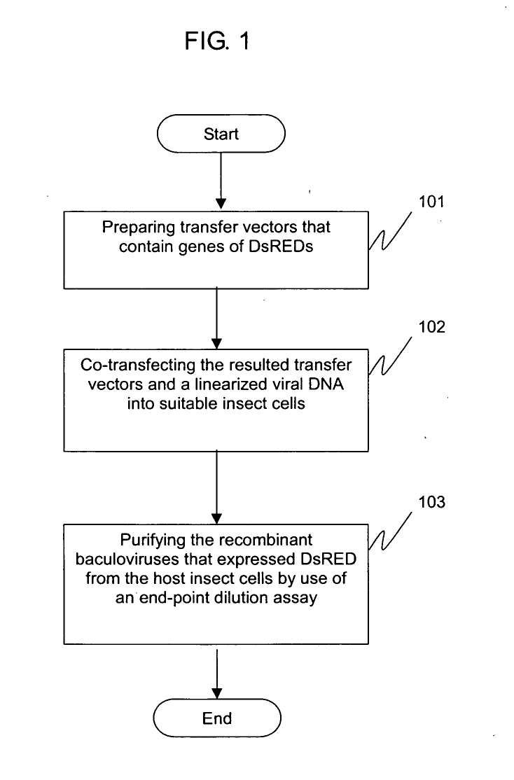

A method of preparing recombinant baculoviruses that expressed DsRED of this invention is illustrated in the flowchart of FIG. 1. Briefly, In step 101, transfer vectors, i.e., DNA constructs containing genes of DsREDs, were prepared according to procedures well known in this art, then the obtained transfer vectors was co-transfected with linearized viral DNA into suitable insect cells such as sf9 cells (step 102), and finally, recombinant baculoviruses that expressed DsRED were purified from the host insect cells by end-point dilution assay (step 103). The end-point dilution assay is a well-known standardized assay. Please see http://www.bdbiosciences.com/clontech/expression/adeno/adeno17.shtml for step-by-step direction of this assay.

Reference will now be made in detail to the present preferred embodiments of the invention, examples of which are illustrated in the accompanying drawings.

### EXAMPLE 1

### Construction of Recombinant Viruses Containing Bicistronic DNA Constructs for Expression of Dual Fluorescence Proteins

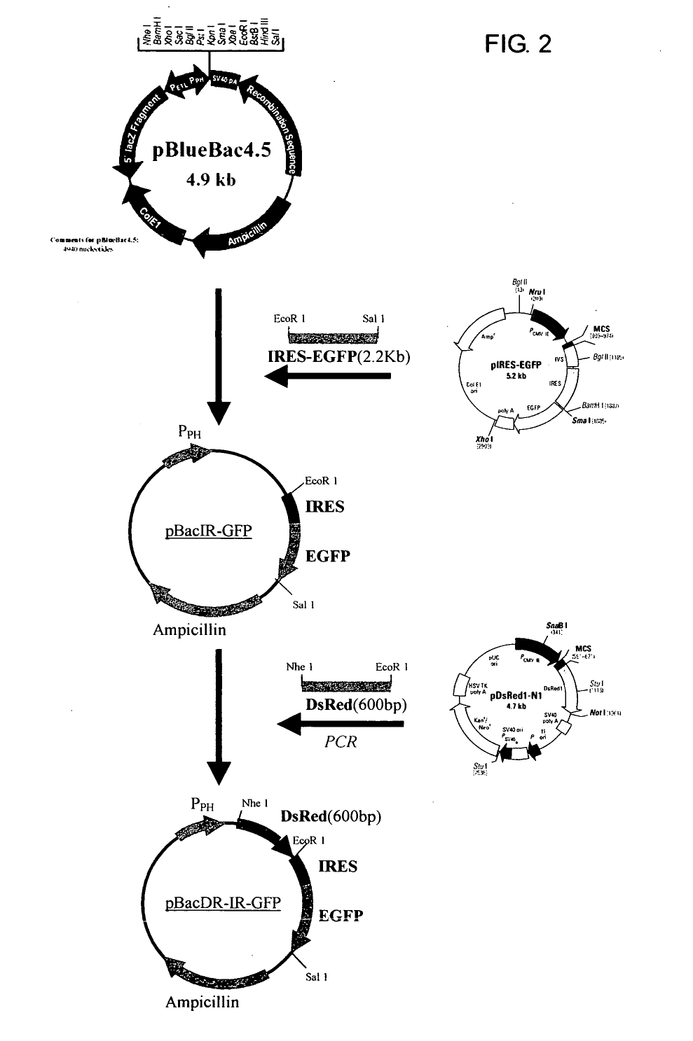

Plasmid pBacDR-IR-GFP is constructed by inserting into the plasmid pBlueBac4.5 (obtained from Invitrogen, Carlsbad, Calif.) with sequences of two genes (cistrons), i.e., coral red fluorescence protein (DsRED), and enhanced green fluorescence protein (EGFP), with an EMCV-IRES sequence between the first cistron (i.e., DsRED) and the second cistron (i.e., EGFP). Briefly, the pIRES-EGFP plasmid (obtained from ClonTech, USA) was digested with EcoRI and Sal, and the 2.2 kb IRES-EGFP DNA fragment was sub-cloned into AcMNPV transfer vector pBlueBac4.5. The resulting plasmid was named pBacIR-GFP. The DsRED gene from the plasmid pDsRED1-N1 (obtained from ClonTech) was PCR amplified with primers and resulted in a DNA fragment containing Nhe1 restriction site on 5′ end and EcoR1 restriction site on 3′ end (the sequence of the primers are as follows and the restriction site is underlined: 5′Nhe1 ATCGGCTAGCGGTCGCCACCATGGTGCGCTCT, 3′ EcoR1 GTAGGAATTCGCTACAGGAACAGGTGGTGG). The PCR amplified DNA fragment was cloned into the Nhe1 and EcoR1 site of the transfer vector pBacIR-GFP and the resulting plasmid was named pBacDR-IR-GFP. FIG. 2 illustrates the DNA organization of the EMCV-IRES based bicistronic DNA constructs containing sequences encoded both DsRED and EGFP.

Bicistronic DNA constructs thus obtained, i.e., pBacDR-IR-GFP, was then cotransfected with linearized viral DNA, Bac-N-Blue (obtained from Invitrogene) in sf9 insect cells, and recombinant viruses, vBAc-DR-IR-GFP, were obtained by end-point dilution assay.

### EXAMPLE 2

### Identification of Insect Cells and/or Larvae Infected with Recombinant Viruses of Example 1

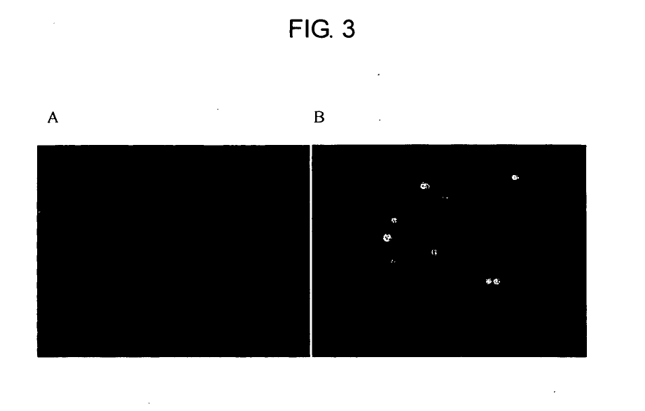

Insect Sf9 cells were infected with the recombinant viruses of Example 1, i.e., vBAc-DR-IR-GFP, and both green fluorescence (FIG. 3A) and red fluorescence (FIG. 3B) can be seen under fluorescence microscope after infection of about 72 hours. This result indicated that the strong polyhedron promoter of AcMNPV could transcribe the two fluorescence protein genes of the bicistronic DNA construct of Example 1, particularly, DsRED is translated by CAP dependent translation mechanism and EGFP is translated by IRES dependent manner.

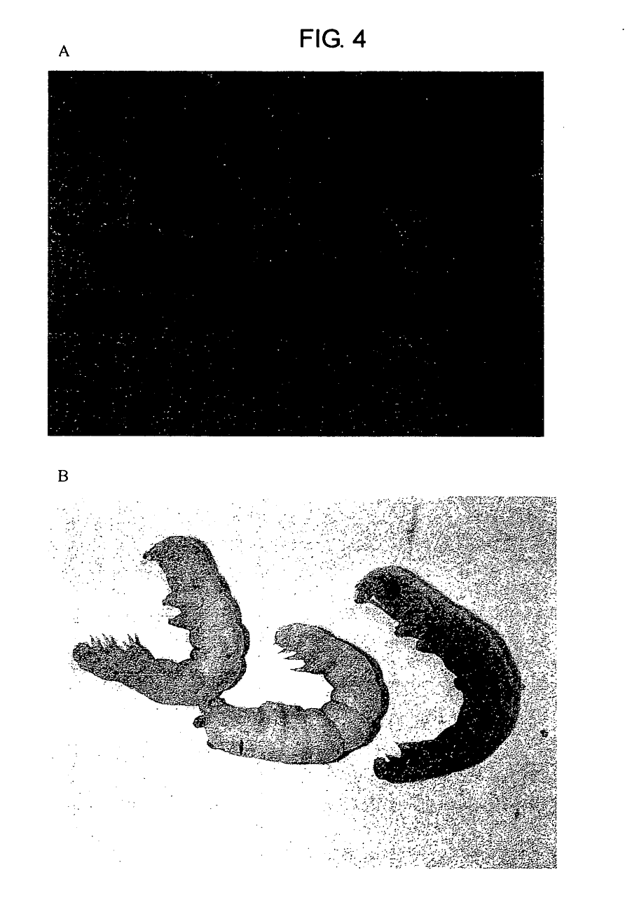

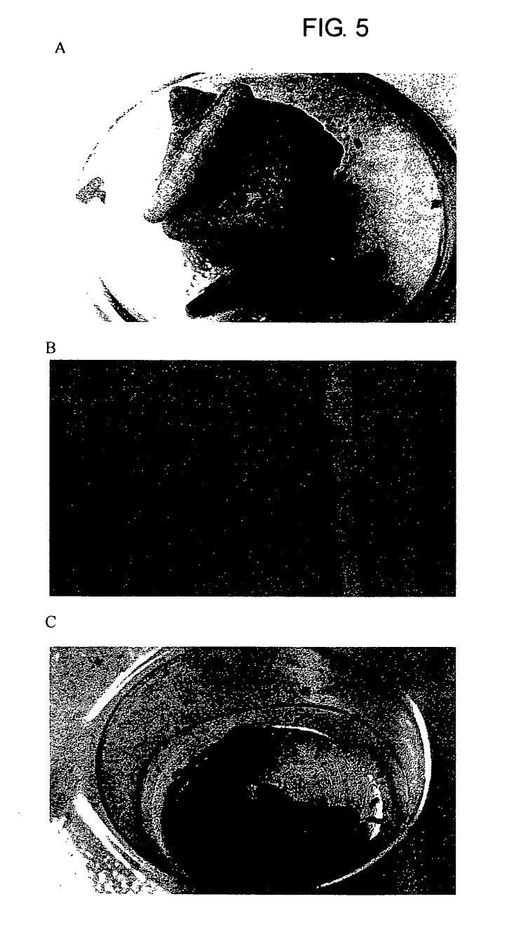

The dual expression of DsRED and EGFP was further examined in insect larvae. Infection of insect larvae was achieved by microinjecting third-instars Trichoplusia ni (T. ni) larvae with vBAc-DR-IR-GFP or vAcp10-G. vAcp10-G is a recombinant AcMNPV containing only the EGFP gene under the control of its p10 promoter, which is constructed in a similar manner as described in Example 1. As expected, the vAcp10-G infected larvae emitted green fluorescence when excited with long-wavelength (365 nm) ultraviolet light (FIG. 4A, the larva on the left), however, said green fluorescence is invisible to the naked eyes under direct sunlight (FIG. 4B, the larva on the left). While most vBAc-DR-IR-GFP infected larvae emitted red fluorescence (FIG. 4A, the larva on the right), few of them appeared yellowish under ultraviolet light excitation (FIG. 4A, the larva in the middle), which probably resulted from the merged of dual fluorescence signals of the evenly expressed and excited DsRED proteins and EGFP proteins. Intriguingly, when viewed under direct sunlight, the green fluorescence emitted from the vAcp10-G infected larvae became faint light green (FIG. 4B, the larva on the left) while the red or yellow fluorescence of vBAc-DR-IR-GFP infected larva appears to be bright pink-red or light pink color, respectively (FIG. 4B, middle and right). Similar results were also observed with third-instars Spodoptera litura larvae (FIG. 5A), Plutella xylostella (FIG. 5B) and Spodoptera exigua larvae (FIG. 5C), respectively. These larvae were inoculated with vBAc-DR-IR-GFP; and the infected larvae emitted pink-red fluorescence under sunlight while the uninfected larvae appeared in dark brown color (FIG. 5). Furthermore, the pink-red fluorescence of the infected larvae can be easily seen by naked eyes without the aid of any prosthetic tools under direct sunlight, which renders the DsRED protein a powerful tracer for effectively tracing and/or monitoring the presence of GMBVs as an insecticide in the field without having the need to perform any tedious molecular analysis.

### INDUSTRAIL APPLICABILITY

The method of the present invention addresses the current tracking problem of GMBVs by co-expression coral red fluorescence proteins as tracers in toxin gene included GMBVs, said expressed coral red fluorescence proteins are bright enough to be seen by naked eyes under direct sunlight, thereby enabling the GMBVs infected pest insects being easily distinguished from those unaffected pest insects. The method according to this invention will ease the public concern of the consequences if the toxin gene included GMBVs were released into the field.

The foregoing description of various embodiments of the invention has been presented for purpose of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method of tracking the presence of genetic modified baculoviruses (GMBVS) in insects, comprising:
infecting the insects with GMBVs that are engineered to express coral red fluorescence proteins; wherein the color of said coral red fluorescence proteins are bright enough to be seen by naked eyes under direct sunlight without an aid of any prosthetic tool.

2. The method of claim 1, wherein said coral red fluorescence proteins are obtained from Discosoma sp.

3. The method of claim 1, wherein said GMBVs contain toxin genes.

4. The method of claim 1, wherein said GMBVs are used as insecticides.

5. The method of claim 1, wherein said infecting comprises microinjecting, feeding, or spraying of a virus fluid containing GMBVs to the insects.

6. The method of claim 5, wherein said infecting comprises spraying of a virus fluid containing GMBVs to the insects.

7. The method of claim 1, wherein said GMBVs infected insects may appear in either red or pink color under direct sunlight.

8. The method of claim 1, wherein said GMBVs infected insects can be easily distinguished from those uninfected insects by their bright colors under direct sunlight due to the expression of said coral red fluorescence proteins.
